(19) United States
(12) Patent Application Publication
(10) Pub. No.: US 2004/0014701 A1
(43) Pub. Date: Jan. 22, 2004

(54) Inhibiting retrotransposon and retroviral integration by targeting the atm pathway

(76) Inventors: Mark James O'Connor, Cambridge (GB); Stephen Philip Jackson, Coton, Cambridge (GB); Alan Lau, Cambridge (GB)

Correspondence Address: BOZICEVIC, FIELD & FRANCIS LLP, 200 MIDDLEFIELD RD, SUITE 200, MENLO PARK, CA, 94025, US

(21) Appl. No.: 10/296,845

(22) PCT Filed: May 30, 2001
(86) PCT No.: PCT/GB01/02398

(51) Int. Cl. A61K 48/00; C12Q 1/70; C12Q 1/68

(52) U.S. Cl. 514/44; 435/5; 435/6

(57) ABSTRACT

Ataxia telangiectasia mutated (ATM)-dependent DNA damage signalling mechanisms are involved in retroviral and retrotransposon integration. Screening methods for inhibitors of retroviral and retrotranspons activity comprise inhibiting the ATM-dependent DNA damage signalling pathway, e.g. by disrupting interaction between components of the pathway. Inhibitors are useful as anti-retroviral agents, e.g. in inhibition of HIV.

The present invention relates to modulating, especially inhibiting processes whereby retroviruses and retrotransposons (retroposons) insert their genetic material into the genome of a eukaryotic host cell in order to carry out a productive infection cycle. More specifically, it relates to proteins of the host cell that have now been found to be required for efficient retrotransposition, which are highly conserved throughout the eukaryotic kingdom but which are not required for cell functioning under most normal conditions. These proteins represent novel targets for anti-retroviral drugs. In addition, assay systems are provided with which anti-retroviral drugs can be screened and tested in vivo and in vitro.

The invention is based on the surprising discovery that ataxia telangiectasia mutated (ATM)-dependent DNA repair and damage signalling mechanisms are involved in retroviral integration. Retrovirus activity is shown by experimental work described herein to be inhibited in mammalian cells where the activity of proteins from the ataxia telangiectasia mutated (ATM)-dependent DNA damage signalling pathway is reduced.

Retroviruses are RNA viruses that must insert a DNA copy (cDNA) of their genome into the host chromosome in order to carry out a productive infection. When integrated, the virus is termed a provirus (Varmus, 1988). Some eukaryotic transposable DNA elements are related to retroviruses in that they transpose via an RNA intermediate. These elements, termed retrotransposons or retroposons, are transcribed into RNA, the RNA is copied into double-stranded (ds) DNA, then the dsDNA is inserted into the genome of the host cell.

The available evidence indicates that the integration of retroviruses and retrotransposons occurs through similar mechanisms, and that retroviruses can be viewed as retrotransposons with an extracellular phase of their life cycle. For example, the Ty1 and Ty5 retrotransposons of the yeast Saccharomyces cerevisiae have been shown to integrate into the host yeast genome by the same type of mechanism that is employed by mammalian retrotransposons and retroviruses to integrate into mammalian host cell DNA (Boeke et al., 1985; Garfinkel, 1985; Grandgenett and Mumm, 1990; Boeke and Sandmeyer, 1991).

Retroviruses are of considerable risk to human and animal health, as evidenced by the fact that retroviruses cause diseases such as acquired immune deficiency syndrome (AIDS; caused by human immunodeficiency virus; HIV-1), various animal cancers, and human adult T-cell leukaemia/lymphoma (Varmus, 1988); also retroviruses have been linked to a variety of other common disorders, including Type I diabetes and multiple sclerosis (Conrad et al., 1997; Perron et al. 1997 and Benoist and Mathis, 1997). In many but not all cases, cancer formation by certain animal retroviruses is a consequence of them carrying oncogenes. Furthermore, retroviral integration and retrotransposition can result in mutagenic inactivation of genes at their sites of insertion, or can result in aberrant expression of adjacent host genes, both of which can have deleterious consequences for the host organism. Retroviruses are also becoming more and more commonly used for gene delivery and are likely to play increasingly important roles in gene therapy. An understanding of how retroviruses function and how they can be controlled is therefore of great commercial and medical importance.

Over recent years, a vast amount of effort has been directed towards identifying inhibitors of retroviral infection because these agents have potential use in combatting retrovirally-borne diseases. To date, most drug development programmes have focussed on virally-encoded products. However, given the short life cycle of retroviruses and their inherently high rates of genetic change, it is clear that a frequent problem with such strategies will be that drug resistant virus derivatives will arise through alterations of the virally-encoded target molecule (for example, Sandstrom and Folks, 1996 and references therein). Thus, most anti-retroviral drugs that interfere with virally-encoded proteins may only have a limited useful life-span. Another limitation of drugs that target virus proteins is that many will not have a broad applicability and will be inherently highly specific to a particular virus or even a certain strain of a particular virus.

Given what is known about the retroviral life cycle, an attractive target for anti-retroviral therapeutics is to interfere with the integration of the double stranded viral cDNA into the host genome. This is an essential step in the retrovirus life cycle and is required for both efficient expression of progeny virus and for the ability of these viruses to cause disease in the infected host (for example, Sakai et al., 1993; for reviews, see Varmus, 1988; Grandgenett and Mumm, 1990). As such, retroviral integration represents an attractive target for novel chemotherapy.

In light of these and other considerations, retroviral reverse transcriptases and integrases have been targeted for drug development. Although this has met with some success, in the case of reverse transcriptases, high rates of genetic change by the targeted virus and variations between different viral strains is likely to limit the scope for anti-reverse transcriptase and anti-integrase drugs, particularly in the long term. As an example of this, two inhibitors of the HIV-1 IN protein that demonstrated in vivo activity were recently described (Hazuda et al, 2000). However, as has been observed with the targeting of other retroviral proteins, exposure of the virus to these drugs in cell culture models resulted in the rapid selection of drug-resistant variants of HIV-1 (Hazuda et al, 2000).

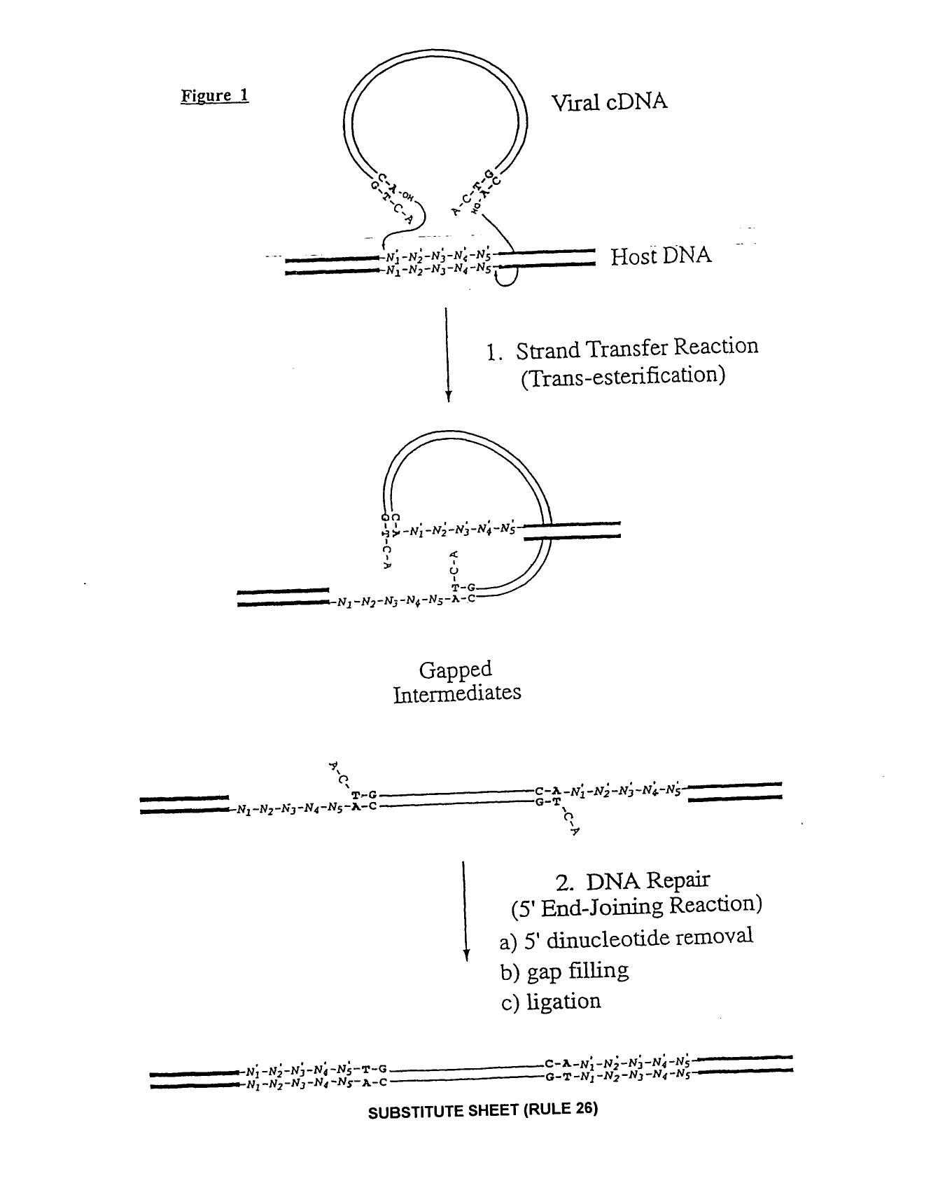

The process of integration involves the coordinated cleavage of double strand DNA breaks in the target sequence and the concomitant attachment of processed viral DNA ends to the host sequences (see Brown, 1990 for review and FIG. 1). The retrovirally-encoded integrase (IN) protein processes each retroviral DNA end to yield 3′hydroxyl ends (—OH) with dinucleotide overhangs (—CA). IN then catalyses the strand transfer reaction, which is a concerted cleavage-ligation (trans-esterification) reaction, covalently joining the viral DNA to host cell DNA. The retroviral integrase (IN) protein therefore has both endonuclease and DNA-strand joining activities. The resulting intermediate contains single stranded DNA gaps with dinucleotide overhangs. This damaged DNA must then be processed and repaired by host cell proteins to complete the integration process.

Detailed analysis of this process with purified IN protein has resulted in the recapitulation of these events in vitro (Katz, 1990; Craigie et al., 1990; Bushman, 1990) and in the development of in vitro assays to identify inhibitors of IN (Chow, 1997; Pommier, 1999).

The final steps of retroviral integration, namely the repair of gapped intermediates, are thought to be dependent upon host DNA repair enzymes. Very little is known about the mechanism of this repair process or even which host enzymes are involved. Interestingly, many DNA repair proteins have been found to be dispensable for normal cell function and long term survival in animal models (for review, see Freidberg 1995). Consequently, the identification of host cell DNA repair proteins involved in retroviral integration would not only increase our understanding of the mechanisms involved in the integration process itself, but could also provide a potential new source of targets for antiretroviral therapies

Until recently, the idea of there being a host factor (or host factors) that is required for retroviral integration but is not necessary for normal host cell growth seemed unlikely. This was because several lines of research have indicated that all the steps needed for covalently linking retrovirus or retrotransposon cDNA to the target DNA molecule can be performed in vitro by purified retroviral integrase protein (for example, Craigie et al., 1990; Bushman et al., 1990; Katz et al., 1990; Grandgenett and Mumm, 1990). In addition, although host factors have been conceived to help with viral integration, it was assumed that these would correspond to “housekeeping proteins” that are essential for host cell viability. Thus, if host “helper” proteins did exist, it was expected that inhibiting them with drugs would not be worthwhile in a therapeutic context because this would also kill the cells of the host.

The first host DNA repair protein implicated in retroviral integration was the nuclear enzyme poly(ADP-ribose) polymerase (PARP) (Gaken et al., 1996). PARP is a zinc finger protein capable of binding double-strand or single strand DNA breaks and catalyzing the attachment of the ADP-ribose moiety of its substrate NAD to suitable acceptors, including PARP itself. It has been proposed that the auto-modification of PARP leads to a dissociation from DNA providing access for components of other DNA repair systems (for review see Lindahl 1995). One important consequence of PARP activation that has been proposed is the prevention of inappropriate homologous or non-homologous recombination promoted by the presence of free DNA strand breaks (Satoh et al., 1994). If this model is correct, then it follows that inhibition of PARP activity would impede the release of PARP from DNA, thus restricting access to other DNA repair enzymes and blocking efficient ligation. Gaken and colleagues were able to show that inhibition of PARP activity through a variety of means resulted in a decrease in retroviral infection and that this was most likely due to a specific effect on integration (Gaken et al., 1996).

A role for Ku and associated proteins has recently been reported in retroviral integration and the mechanistically related process of retrotransposition (Daniel et al., 1999; Downs and Jackson, 1999; WO/GB98/00099). Ku is a heterodimer of proteins of ˜70 and 80 kDa (Ku70 and Ku80 respectively), which together with the DNA-dependent protein kinase (DNA-PK) catalytic subunit, plays a pivotal role in double stranded break (DSB) repair through the DNA non-homologous end-joining (NHEJ) pathway (for review see Critchlow and Jackson, 1998). Ku has been shown to be associated with the virus-like particles (VLPs) of the yeast retrotransposable element Ty1, and potentiates retrotransposition (Downs and Jackson, 1999). Other components of the Ku-associated DNA repair pathway, such as DNA-PKcs and XRCC4 (a NHEJ protein that is thought to recruit DNA ligase IV to DNA DSBs) have also been shown to be required for efficient retroviral integration in mammalian cells (Daniel et al., 1999). While these two studies suggest a role for the Ku-associated pathway in retroviral integration, the requirement is not absolute and residual integration events are detected in all cases (Downs and Jackson, 1999; Daniel et al., 1999).

Investigations into the role of host cell DNA repair associated proteins in retroviral integration are described in the present application. The findings herein provide indication that the ataxia telangiectasia mutated (ATM)-dependent DNA damage signalling pathway plays a role in the retroviral integration process, and opens up new opportunities for anti-retroviral action. Experimental data herein includes inhibition of HIV activity. HIV is a preferred retroviral target in many aspects and embodiments of the present invention.

According to one aspect of the present invention, there is provided a method of inhibiting retrovirus and/or retrotransposon activity by means of a substance identified as an inhibitor of ataxia telangiectasia mutated (ATM)-dependent DNA damage signalling.

Methods of treatment of the human or animal body by way of therapy may be excluded. Thus, for example, a method may be carried out in vitro or ex vivo, e.g. on transplant material, or may be used to treat a plant.

However, a further aspect of the present invention provides the use of a substance identified as an inhibitor of the ataxia telangiectasia mutated (ATM)-dependent DNA damage signalling pathway in the manufacture of a medicament for inhibiting retrovirus and/or retrotransposon activity.

Another aspect of the present invention provides a substance identified as an inhibitor of the ataxia telangiectasia mutated (ATM)-dependent DNA damage signalling pathway for use in inhibiting retrovirus and/or retrotransposon activity.

A further aspect of the present invention provides the use of a substance identified as an inhibitor of the ataxia telangiectasia mutated (ATM)-dependent DNA damage signalling pathway in inhibiting retrovirus and/or retrotransposon activity.

The substance may be provided in a composition which includes at least one other component, for instance a pharmaceutically acceptable excipient, as discussed further below.

The substance may be provided in vivo to cells in a human or animal body, by way of therapy (which may include prophylaxis), or in planta, ex vivo or in vitro. This too is discussed further elsewhere herein. Suitable substances may include peptide fragments of ATM-dependent DNA damage signalling pathway components, such as peptide fragments of ATM, Chk1, Chk2, NBS1, Rad50, Mre11, BRCA1, p53 or p53R2.

Integration of a retrovirus and/or retrotransposon into the genome of a cell may be inhibited by treatment of the cell with a substance which is an inhibitor of the ataxia telangiectasia mutated (ATM)-dependent DNA damage signalling pathway.

Aspects of the present invention may exclude wortmannin as an inhibitor of the ataxia telangiectasia mutated (ATM)-dependent DNA damage signalling pathway.

Inhibition of proteins from the ataxia telangiectasia mutated (ATM)-dependent DNA damage signalling pathway may be achieved in any of numerous different ways, without limitation to the nature and scope of the present invention.

In certain embodiments of the present invention, ATM itself is targeted for inhibition, that is to say that ATM's involvement in the ATM-dependent DNA damage signalling pathway is inhibited in order to inhibit ATM-dependent DNA damage signalling and thereby retroviral integration. One way, therefore, of inhibiting ATM activity is to use a substance that inhibits ATM kinase activity or the interaction of ATM with either DNA or with another component of the ATM-dependent DNA damage signalling pathway. Otherwise, ATM itself need not be targeted and the function or activity of one or more other components of the ATM-dependent DNA damage signalling pathway may be inhibited (discussed further below). Of course, a substance may inhibit activity of a component of the pathway such as ATM not (or not solely) by inhibiting physical interaction between the component and another but by binding at an active site or by binding in a way that has a steric effect on the conformation of an active site and thus activity of the component. Precisely how the activity or function of a component of the pathway is inhibited need not be relevant to practising the present invention.

The nucleic acid and protein sequences of various components of the ATM-dependent DNA damage signalling pathway in humans and yeast are available from the GenBank database, under the following accession numbers: Human ATM (Nucleic acid coding sequence (CDS): W82828, Protein sequence: AAB65827, Human Chkl (CDS: AF016582, Protein: AAC51736), Human Chk2 (CDS: NM_{—}007194, Protein: 096017), NBS1 (CDS: AF3169124, Protein: BAA28616), Human Rad50 (CDS: 5032016, Protein: NP_{—}005723), Mrell (CDS: U37359, Protein: AAC78721), BRCA1 (CDS: U14680, Protein: A58881), Human p53 (CDS: AH007667, Protein: AAD28628) and Human p53R2 (AB036063, AB036524-AB036532).

The activity or function of a component of the ATM-dependent DNA damage signalling pathway (such as ATM) may be inhibited, as noted, by means of a substance that interacts in some way with the component. An alternative employs regulation at the nucleic acid level to inhibit activity or function by down-regulating production of the component.

For instance, expression of a gene may be inhibited using anti-sense technology. The use of anti-sense genes or partial gene sequences to down-regulate gene expression is now well-established.

Anti-sense oligonucleotides may be designed to hybridise to the complementary sequence of nucleic acid, pre-mRNA or mature mRNA, interfering with the production of a component of the ATM-dependent DNA damage signalling pathway, such as ATM, encoded by a given DNA sequence, so that its expression is reduced or completely or substantially completely prevented. In addition to targeting coding sequence, antisense techniques may be used to target control sequences of a gene, e.g. in the 5′ flanking sequence, whereby the antisense oligonucleotides can interfere with expression control sequences. The construction of antisense sequences and their use is described for example in Peyman and Ulman, Chemical Reviews, 90:543-584, (1990) and Crooke, Ann. Rev. Pharmacol. Toxicol., 32:329-376, (1992).

Oligonucleotides may be generated in vitro or ex vivo for administration or anti-sense RNA may be generated in vivo within cells in which down-regulation is desired.

Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of antisense inhibition. It may be advantageous to include the initiating methionine ATG codon, and perhaps one or more nucleotides upstream of the initiating codon. A suitable fragment may have about 14-23 nucleotides, e.g. about 15, 16 or 17.

Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992, and Molecular Cloning: a Laboratory Manual: 2nd edition, Sambrook et al., 1989, Cold Spring Harbor Laboratory Press.

Another possibility is that nucleic acid is used which on transcription produces a ribozyme, able to cut nucleic acid at a specific site—thus also useful in influencing gene expression. Background references for ribozymes include Kashani-Sabet and Scanlon, 1995, Cancer Gene Therapy, 2(3): 213-223, and Mercola and Cohen, 1995, Cancer Gene Therapy, 2(1), 47-59.

It is well known that pharmaceutical research leading to the identification of a new drug may involve the screening of very large numbers of candidate substances, both before and even after a lead compound has been found. This is one factor which can make pharmaceutical research very expensive and time-consuming. Means for assisting in the screening process can have considerable commercial importance and utility. Such means for screening for substances potentially useful in inhibiting retroviral and/or retrotransposon activity is provided according to the present invention. Substances identified as modulators of ATM-dependent DNA damage signalling represent an advance in the fight against retroviral diseases (for instance), since they provide basis for design and investigation of therapeutics for in vivo use.

A method of screening for a substance which inhibits retrovirus and/or retrotransposon activity may include contacting one or more test substances with one or more components of the ATM-dependent DNA damage signalling pathway of an organism of interest in a suitable reaction medium, and testing for substance/component interaction, e.g. by assessing activity of the ATM-dependent DNA damage signalling pathway or component thereof and comparing that activity with the activity in comparable reaction medium untreated with the test substance or substances. A difference in activity between the treated and untreated samples is indicative of a modulating effect of the relevant test substance or substances. It may be sufficient, at least as a preliminary, to only assess physical interaction between test substance and pathway component or subunit thereof in test samples, rather than actual biochemical activity.

In further aspects, the present invention relates to the screening of candidate substances for potential as inhibitors of retrovirus and/or retrotransposon activity. More particularly, it provides a method by which test substances can be screened for their ability to affect ATM-dependent DNA damage signalling. Test substances may be screened for inhibition or activation of the pathway, though clearly inhibitors of the pathway are of primary interest e.g. for anti-retroviral treatment.

According to a further aspect of the present invention there is provided a method of screening for a substance which is an inhibitor of retrovirus and/or retrotransposon activity, particularly events leading to productive nucleic acid integration or transposition of retrovirus and/or retrotransposon, which includes:

providing an ATM-dependent DNA damage signalling pathway;

exposing the pathway to a test substance under conditions which would normally lead to the activation of the ATM-dependent DNA damage signalling pathway; and

looking for an end-point indicative of activation of the ATM-dependent DNA damage signalling pathway; whereby inhibition of that end-point indicates inhibition of the ATM-dependent DNA damage signalling pathway by the test substance.

The pathway may be provided in a cell to be exposed to the test substance, or the assay may be performed on an in vitro ATM-dependent DNA damage signalling system that measures the accuracy and efficiency of joining together DNA strand breaks that have been created by treating intact DNA with restriction endonucleases, chemicals, or radiation.

Activation of the ATM-dependent DNA damage signalling pathway may be caused by DNA double-strand breaks (DSBs), single strand gaps in the DNA double helix and by other disruptions to the DNA double-helix. These structures exist at the ends of retroviral and retrotransposon DNA and occur as intermediates in the retroviral integration and retrotransposition process. To assay for ATM-dependent DNA damage signalling, retrovirus or retroviral DNA, intermediates in retroviral integration or retrotransposon integration, or synthetic preparations of DNA that mimic any of these may be provided.

The end-point of the screen may be the phosphorylation of downstream target proteins, such as p53. Phosphorylation may be determined by any suitable method known to those skilled in the art. It may be detected by methods employing radiolabelled ATP and optionally a scintillant. By way of example, phosphorylation of a protein may be detected by capturing it on a solid substrate using an antibody or other specific binding molecule directed against the protein and immobilised to the substrate, the substrate being impregnated with a scintillant—such as in a standard scintillation proximity assay. Phosphorylation is then determined via measurement of the incorporation of radioactive phosphate.

Phosphate incorporation into a protein such as p53 may also be determined by precipitation with acid, such as trichloroacetic acid, and collection of the precipitate on a nitrocellulose filter paper, followed by measurement of incorporation of radiolabelled phosphate.

Phosphorylation may be detected by methods employing an antibody or other binding molecule which binds the phosphorylated peptide with a different affinity to unphosphorylated peptide. Such antibodies may be obtained by means of any standard technique as discussed elsewhere herein. Binding of a binding molecule which discriminates between the phosphorylated and non-phosphorylated form of a peptide may be assessed using any technique available to those skilled in the art, examples of which are discussed elsewhere herein.

As other end points for screens, the effect on the repair of DNA damage, or cell viability or apoptosis may be measured. Suitable methods are known to those skilled in the art.

That a substance is inhibitory of the ATM-dependent DNA damage signalling pathway may be verified by hypersensitivity of mammalian cells to ionising radiation (Taylor et al., 1975) or by rejoining of double-strand breaks (e.g. in a plasmid) in vivo (Liang et al., 1996). Biochemical methods, such as PCR or nucleic acid hybridisation/detection methods, may be used, e.g. to detect the chemical structure of integration products. Retroviral integration and/or retrotransposition may be scored for example by detection using standard genetic, biochemical or histological techniques.

It should be noted that in assaying for ability of a test substance to affect an ATM-dependent DNA damage signalling pathway, the end-point chosen to be determined in the assay need not be the actual end-point of the DNA repair pathway (the repair of DNA), but may be the activity which a component of the pathway exhibits in the pathway.

Of course, as noted elsewhere, reference to a component of an ATM-dependent DNA damage signalling pathway may be taken to refer to a derivative, variant or analogue of the relevant component which has the requisite, assayable property or activity (e.g. ability to bind another component in the pathway).

Given the teaching provided herein of the ability to inhibit retroviral and/or retrotransposon activity by manipulating the ATM-dependent DNA damage signalling pathway, those of ordinary skill in the art may design assays for antiretroviral agents by employing proteins or fragments thereof which are homologous with a component of the ATM-dependent DNA damage signalling pathway in the expectation that substances which affect the activity of the homologue will be able to affect the activity of the component.

Prior to, as well as or instead of being screened for actual ability to affect ATM-dependent DNA damage signalling activity, test substances may be screened for ability to interact with a component of the pathway (such as ATM) e.g. in a yeast two-hybrid system (which requires that both the polypeptide component and the test substance can be expressed in yeast from encoding nucleic acid). This may for example be used as a coarse screen prior to testing a substance for actual ability to modulate activity.

Thus, in a further aspect, the present invention provides a method of screening for a substance which is an inhibitor of retrovirus and/or retrotransposon activity, particularly nucleic acid integration of retrovirus and/or retrotransposon, which includes:

providing a component or a fragment thereof of a ATM-dependent DNA damage signalling pathway;

exposing the component to a test substance;

determining interaction between the component and the test substance.

Such a method may include determining ability of a test compound which interacts with said component or fragment thereof to inhibit the ATM-dependent DNA damage signalling pathway, and/or to inhibit retrovirus and/or retrotransposon activity. The component of the ATM pathway may be ATM, Chkl, Chk2, NBS1, Rad50, Mre11, BRCA1, pS3, p53R2 or other components of the ATM-dependent DNA damage signalling pathway.

The present invention also provides a method of screening for an agent which is an inhibitor of retrovirus and/or retrotransposon activity, including:

providing first and second substances, the first substance including a first component of a ATM-dependent DNA damage signalling pathway or a peptide fragment, derivative, variant or analogue thereof able to bind a second component of the ATM-dependent DNA damage signalling pathway, the second substance including the second component of the ATM-dependent DNA damage signalling pathway or a peptide fragment, derivative, variant or analogue thereof able to bind said first component, under conditions in which the components normally interact;

exposing the substances to a test compound;

determining interaction between the two substances in the presence of the test compound.

Such methods may include determining ability of a test compound to inhibit enzymatic activity or which disrupts interaction between the two components, to inhibit the ATM-dependent DNA damage signalling pathway, and/or to inhibit retrovirus and/or retrotransposon activity. Enzymatic activity inhibited by such methods may include kinase, helicase, nuclease, and ribonucleotide reductase (RNRase).

The present invention also encompasses methods which include adding inhibitors obtained by methods described herein to a cell to inhibit retrovirus and/or retrotransposon activity in the cell. Suitable cells may be in vitro cell lines (i.e. not part of the human or animal body).

A yeast two-hybrid system (e.g Evan et al. Mol. Cell. Biol. 5, 3610-3616 (1985); Fields & Song Nature 340, 245-246 (1989)) may be used to identify substances that interact with a ATM-dependent DNA damage signalling pathway component or subunit thereof. This system often utilises a yeast containing a GAL4 responsive promoter linked to β-galactosidase gene and to a gene (HIS3) that allows the yeast to grow in the absence of the amino acid histidine and to grow in the presence of the toxic compound 3-aminotriazole. The pathway component or subunit may be cloned into a yeast vector that will express the protein as a fusion with the DNA binding domain of GAL4. The yeast may then be transformed with DNA libraries designed to express test polypeptides or peptides as GAL4 activator fusions. Yeast that have a blue colour on indicator plates (due to activation of β-galactosidase) and will grow in the absence of histidine (and the presence of 3-aminotriazole) may be selected and the library plasmid isolated. The library plasmid may encode a substance that can interact with the ATM-dependent DNA damage signalling pathway component or subunit thereof.

A variation on this may be used to screen for substances able to disrupt interaction between two components of the ATM-dependent DNA damage signalling pathway, or the subunits of a such a component. For instance, the components or subunits may be expressed in a yeast two-hybrid system (e.g. one as a GAL4 DNA binding domain fusion, the other as a GAL4 activator fusion) which is treated with test substances. The absence of the end-point which normally indicates interaction between the components or subunits (e.g. the absence of a blue colour in the exemplary system outlined above) when a test substance is applied indicates that substance disrupts interaction between the two components or subunits, and may therefore inhibit ATM-dependent DNA damage signalling, indicative of potential as an inhibitor of retrovirus and/or retrotransposon activity.

In assays and screens according to embodiments of the present invention, appropriate control experiments may be performed in accordance with appropriate knowledge and practice of the ordinary skilled person. Experiments may be peformed in the presence and absence of a test compound, substance or agent.

For potential therapeutic purposes, the ATM-dependent DNA damage signalling pathway or one or more components (or subunits) thereof used in the assay may be human, or mammalian or bird bearing in mind veterinary applications. However, given the ease of manipulation of yeast, and the good conservation between ATM-dependent DNA damage signalling components in different eukaryotes (Bentley et al., 1997), an assay according to the present invention may involve applying test substances to a yeast system with the expectation that similar results will be obtained using the substances in mammalian, e.g. human, systems. In other words, a substance identified as being able to inhibit DNA damage signalling of ATM homologues in yeast may be able to inhibit ATM-dependent DNA damage signalling in other eukaryotes. A further approach, as discussed, is to use yeast cells expressing one or more components (e.g. ATM) or subunits of the ATM-dependent DNA damage signalling pathway of another eukaryote, e.g. human. A plant ATM-dependent DNA damage signalling pathway or one or more components thereof may be employed in an assay according to the present invention, to test for substance useful in inhibiting retroviral and/or retrotransposon activity in the plant or plants generally.

Following identification of a substance which modulates or affects ATM-dependent DNA damage signalling and/or interaction between components of the pathway or subunits thereof, the substance may be investigated further, in particular for its ability to inhibit retroviral and/or retrotransposon activity. Furthermore, it may be manufactured and/or used in preparation, i.e. manufacture or formulation, of a composition such as a medicament, pharmaceutical composition or drug. These may be administered to individuals.

Thus, the present invention extends in various aspects not only to a substance identified as inhibiting retroviral and/or retrotransposon activity in accordance with what is disclosed herein, but also a pharmaceutical composition, medicament, drug or other composition comprising such a substance, a method comprising administration of such a composition to a patient, e.g. for treatment (which may include preventative treatment) of a retroviral disorder, use of such a substance in manufacture of a composition for administration, e.g. for treatment of a retroviral disorder, and a method of making a composition comprising admixing such a substance with a pharmaceutically acceptable excipient, vehicle or carrier, and optionally other ingredients.

A substance that tests positive in an assay according to the present invention or is otherwise found to inhibit retroviral and/or retrotransposon activity by inhibition of ATM-dependent DNA damage signalling may be a peptide or peptide fragment or may be non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. peptides are unsuitable active agents for oral compositions as they tend to be quickly degraded by proteases in the alimentary canal. Mimetic design, synthesis and testing are generally used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, eg by substituting each residue in turn. Alanine scans of peptide are commonly used to refine such peptide motifs. These parts or residues constituting the active region of the compound are known as its “pharmacophore”.

Once the pharmacophore has been found, its structure is modelled to according its physical properties, eg stereochemistry, bonding, size and/or charge, using data from a range of sources, eg spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. Alternatively, where the mimetic is peptide based, further stability can be achieved by cyclising the peptide, increasing its rigidity. The mimetic or mimetics found by this approach can then be screened to see whether they have the target property, or to what extent they exhibit it. Further optimisation or modification can then be carried out to arrive at one or more final mimetics for in vivo or clinical testing.

A substance for inhibiting retrovirus and/or retrotransposon activity in accordance with any aspect of the present invention may be formulated in a composition. A composition may include, in addition to said substance, a pharmaceutically acceptable excipient, carrier, buffer, stabiliser or one or more other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at a particular site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

Targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they may be produced in the target cells by expression from an encoding gene introduced into the cells. The vector may be targeted to the specific cells to be treated, or it may contain regulatory elements which are switched on more or less selectively by the target cells.

The agent may be administered in a precursor form, for conversion to the active form by an activating agent produced in, or targeted to, the cells to be treated.

A composition may be administered alone or in combination with other treatments, either simultaneously or sequentially dependent upon the condition to be treated.

The present invention also encompasses methods and assays for determining and quantifying retroviral integration events. Such assays and methods may be used to determine the effect of agents of interest and/or host cell mutations on retroviral integration.

Methods and assays of the present invention may comprise direct detection of a reporter gene within the retrovirus. Reporter genes used for the quantification of retroviral integration events may include antibiotic resistance genes, such as those coding for resistance to Neomycin (G418), Puromycin, or Hygromycin. Alternative reporters include autofluorescent reporters such as the green fluorescent protein GFP or variants thereof or enzymatic gene products such as β-galactosidase, or chloramphenicol acetyl transferase (CAT). However, the preferred reporter gene for retroviral integration assays may consist of genes coding for products capable of generating chemiluminescence. The preferred reporter gene in some embodiments of the invention described here is the firefly (Photinus pyralis) luciferase gene which provides both a high level of sensitivity and as a result of this, an ability to be used in high throughput assays. Alternatives to the firefly luciferase gene would include the Sea Pansy (Renilla reniformis) luciferase gene product.

An assay of retroviral integration into host cells may include;

infecting host cells with retrovirus, said retrovirus containing a reporter gene encoding a chemiluminescent protein,

causing or allowing expression of said reporter gene from integrated retroviruses; and

determining luminescence generated by said chemiluminescent protein.

Host cells may be transduced/infected with retrovirus in the presence or absence of an agent of interest. The effect of the agent of interest on retroviral integration may then be assessed by comparing the luminescent signals produced in the presence and absence of agent. Alternatively, the effect of a host cell mutation on retroviral integration may be determined by comparing the luminescent signals produced by host cells with and without the mutation.

Assays may be conveniently carried out in a 96-well microtitre plate format. Reagents and materials for generating and measuring a luminescent end point are well known in the art and are available commercially. Such reagents and materials may be used by a skilled person in accordance with the manufacturer's instructions as appropriate. The retroviral luciferase integration assay (LUCIA) represents a significant improvement on all current available retroviral integration assays, including the colony formation assay (CFA), which utilises drug resistance markers and takes significantly longer than LUCIA and which is not amenable to High Throughput Screening (HTS), or assays utilising β-galactosidase activity which do not possess the inherent sensitivity of luciferase-based assays.

The experimental basis for the invention and illustrative embodiments of the invention will now be described in more detail, with reference to the accompanying drawings. All publications mentioned anywhere in the text are incorporated herein by reference.

FIG. 1 shows a schematic model for in vivo retroviral DNA integration. The sequences shown correspond to HIV DNA ends.

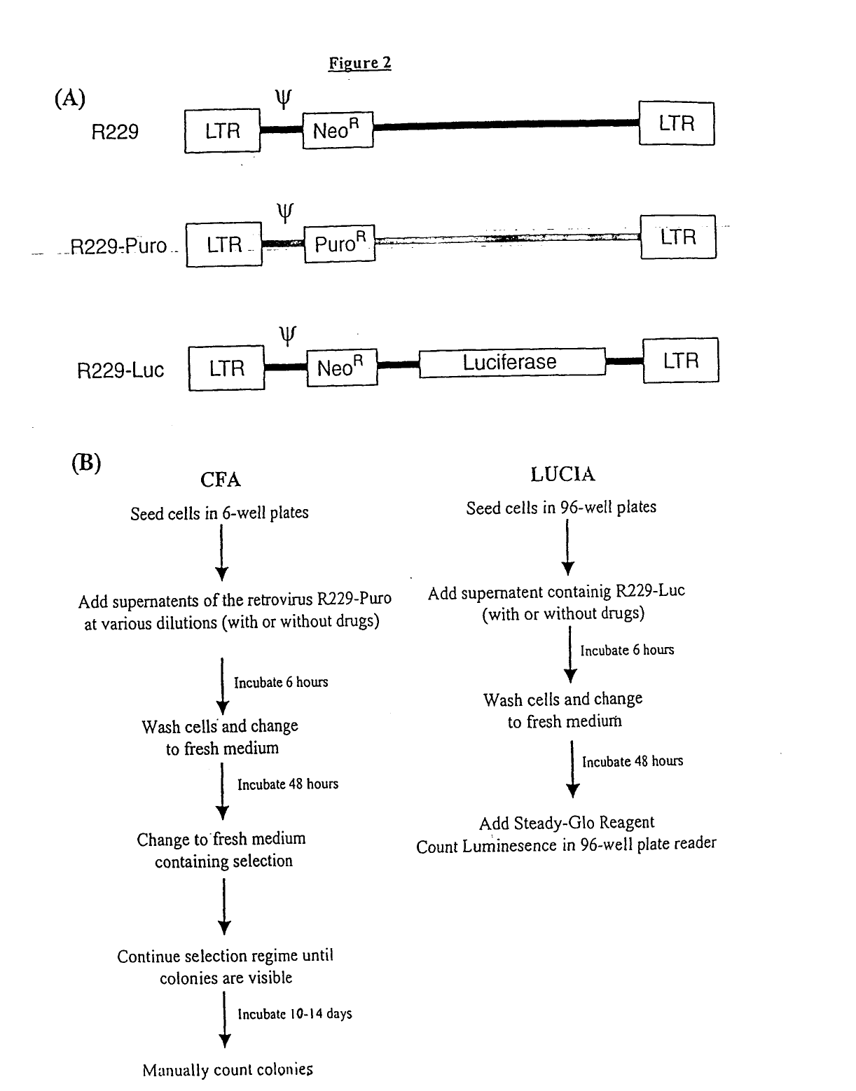

FIG. 2 shows a comparison of the colony formation assay (CFA) and the luciferase integration assay (LUCIA) as methods for determining retroviral integration events.

FIG. 2(A) shows a schematic diagram of the progenitor retroviral vector R229 and the constructed vectors R229-Puro and R229-Luc (see materials and methods) used in the CFA and the LUCIA respectively.

FIG. 2(B) shows a comparison of the methods for CFA and LUCIA in the determination of retroviral integration events.

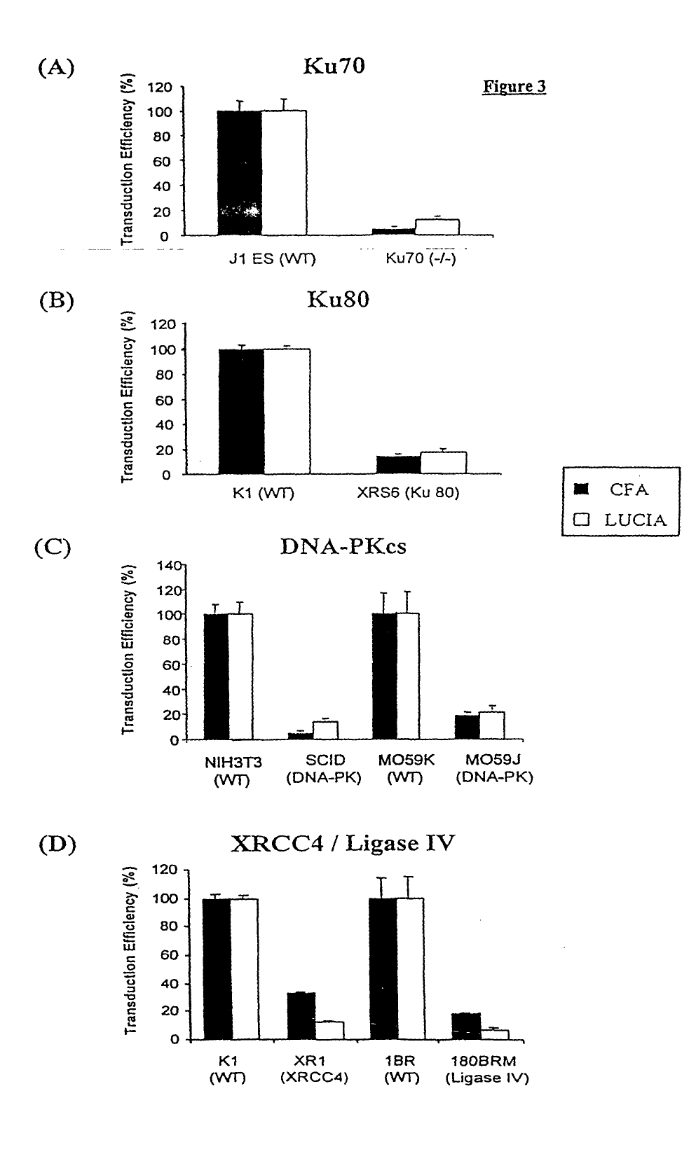

FIG. 3 compares data from LUCIA and CFA assays which show the roles of various proteins (Ku70, Ku80, DNA-PKcs, XRCC4 and DNA ligase IV) from the Ku-associated DNA NHEJ pathway in retroviral integration.

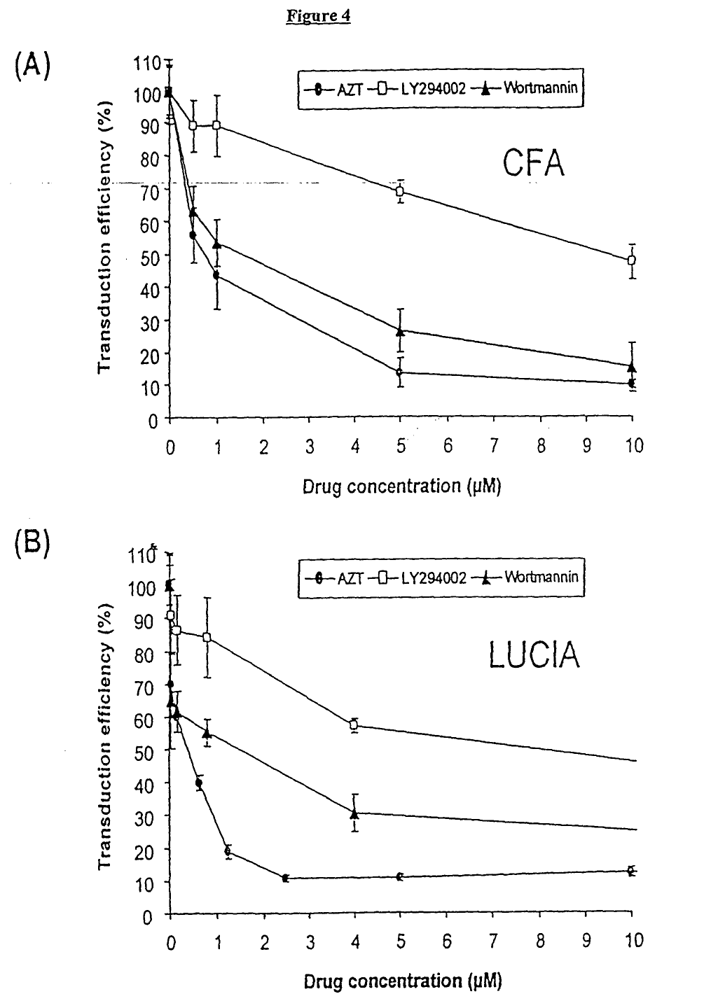

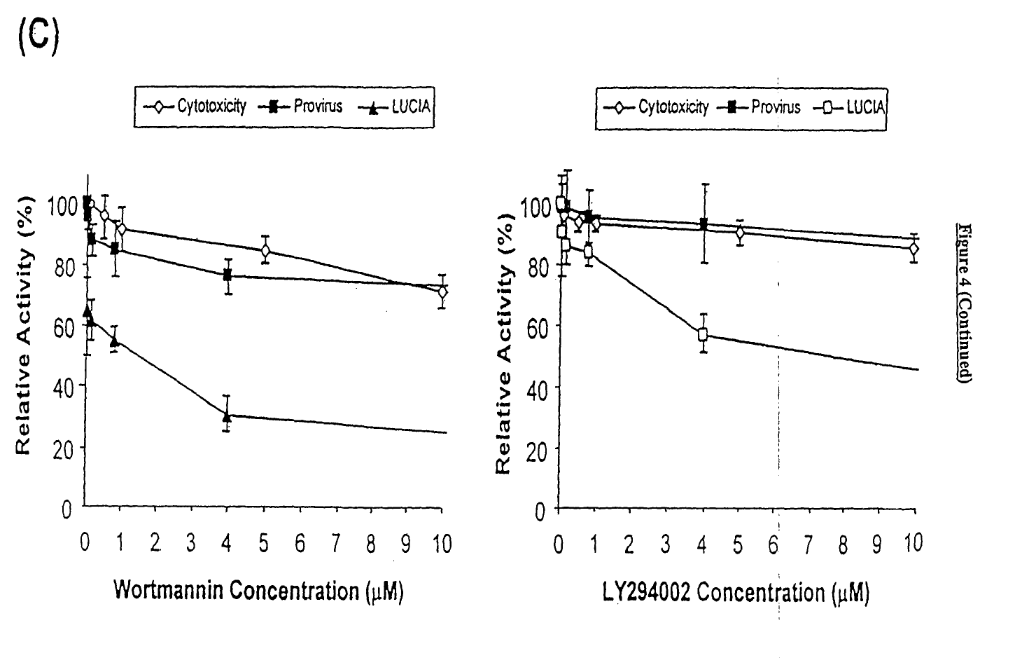

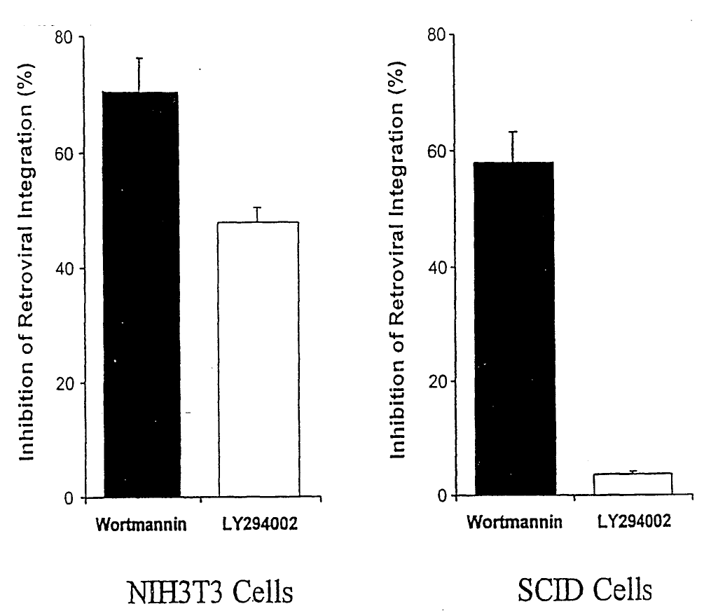

FIG. 4 shows data from CFA and LUCIA assays which show that retroviral integration is abrogated by the DNA-PK inhibitors wortmannin and LY294002.

FIG. 5 shows data from LUCIA assays which show that residual retroviral integration events in DNA-PKcs defective SCID cells can be further inhibited by treatment with wortmannin but not with LY294002.

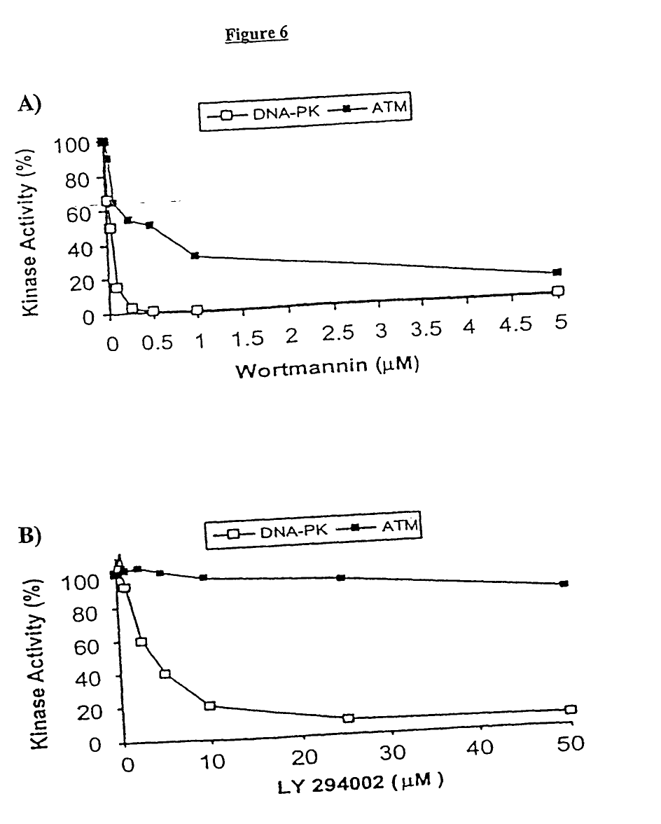

FIG. 6 shows the results of kinase assays which demonstrate the differential inhibition of DNA-PK and ATM kinase activity by wortmannin and LY294002.

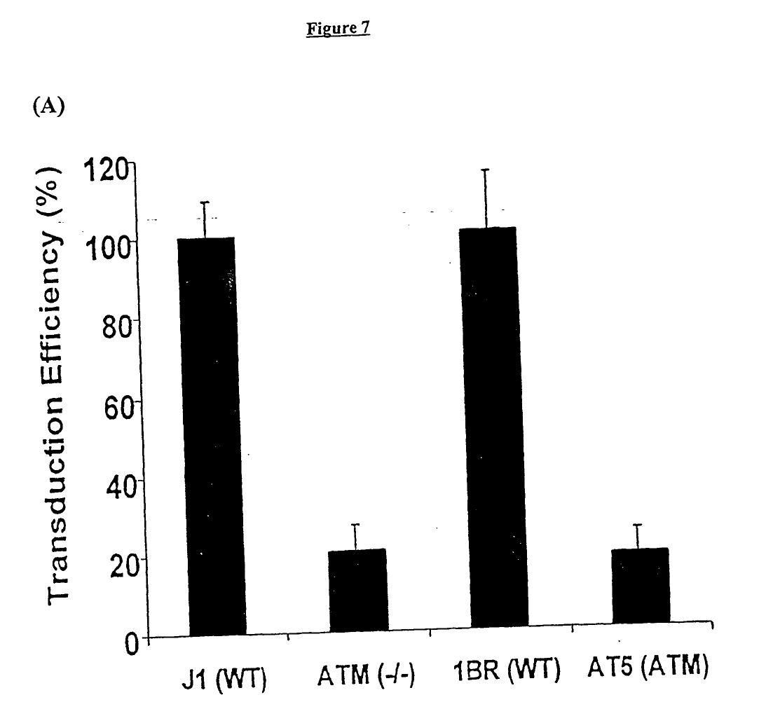

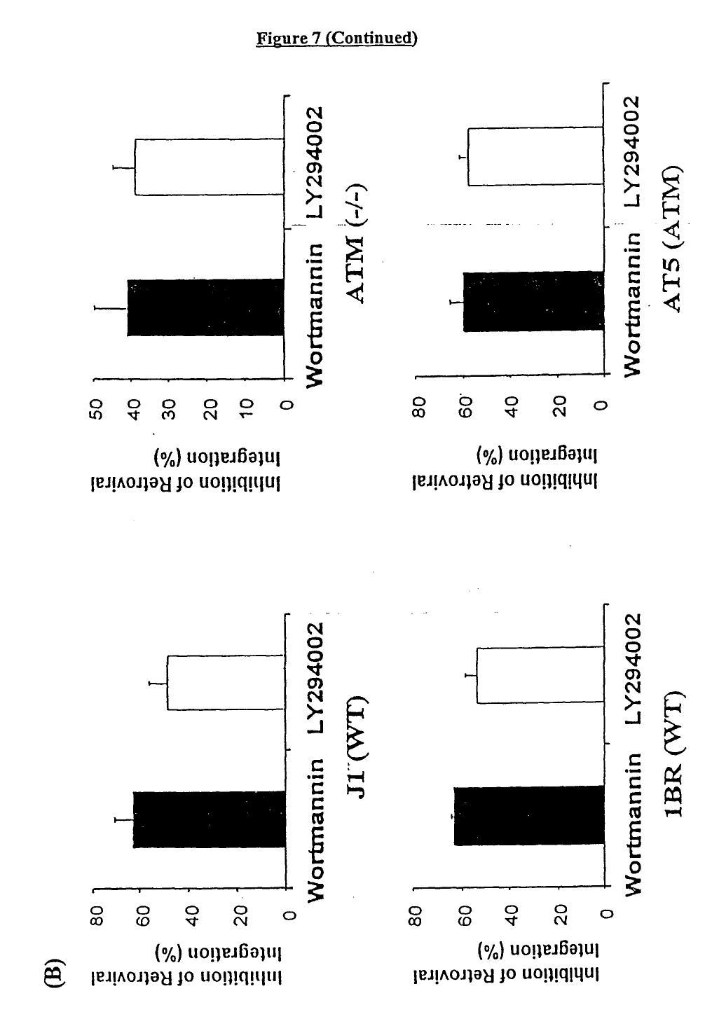

FIG. 7 shows the results of LUCIA assays demonstrating that the ataxia-telangiectasia mutated (ATM) protein plays a critical role in retroviral integration.

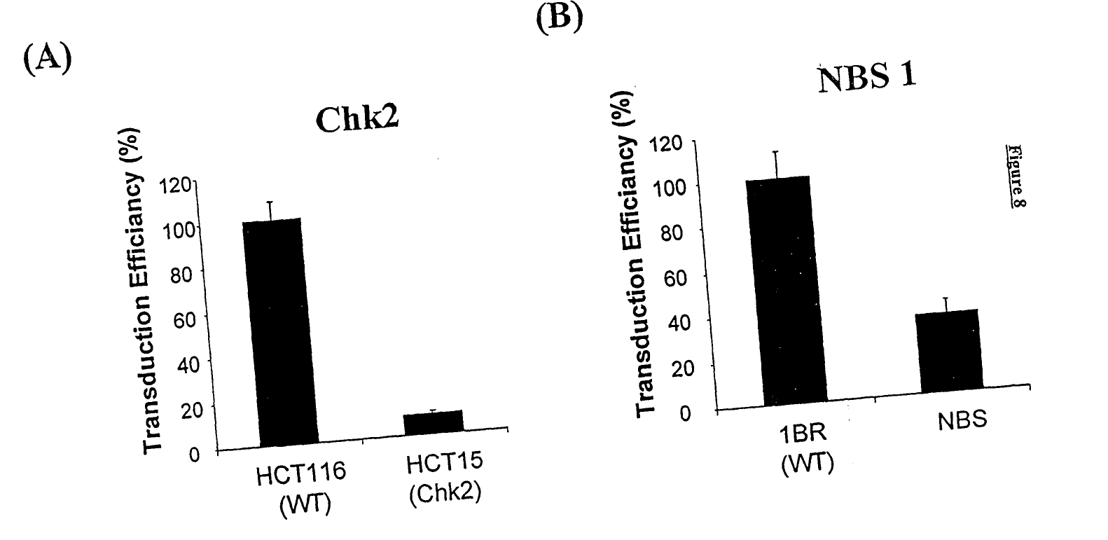

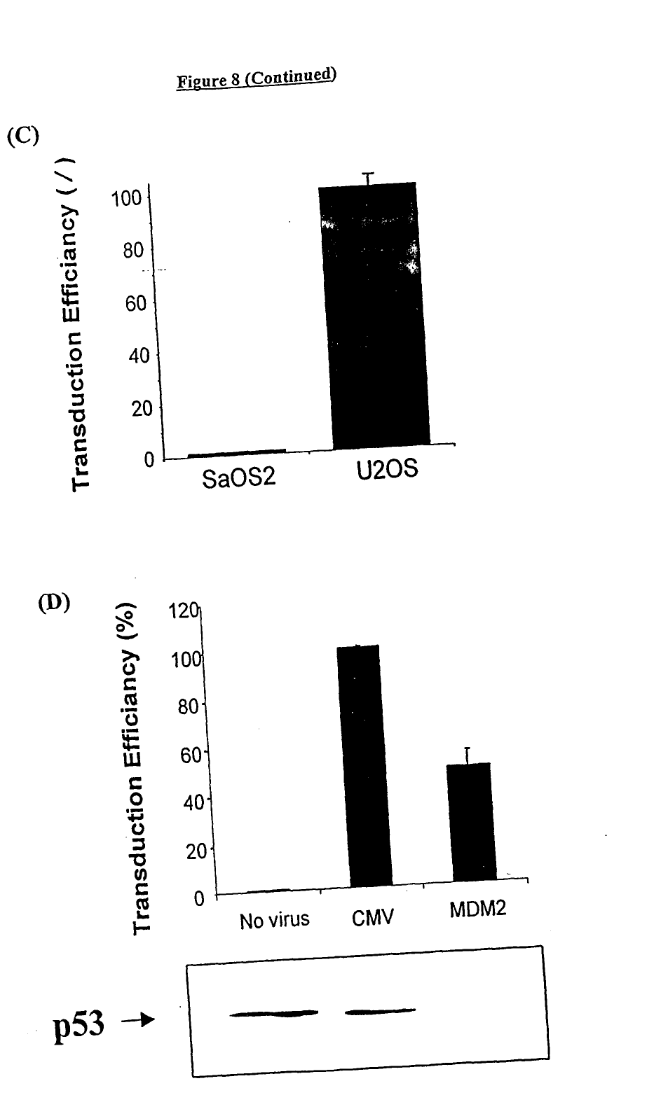

FIG. 8 shows the results of LUCIA assays showing that multiple components of the ATM-associated DNA damage signalling pathway are required for efficient retroviral integration.

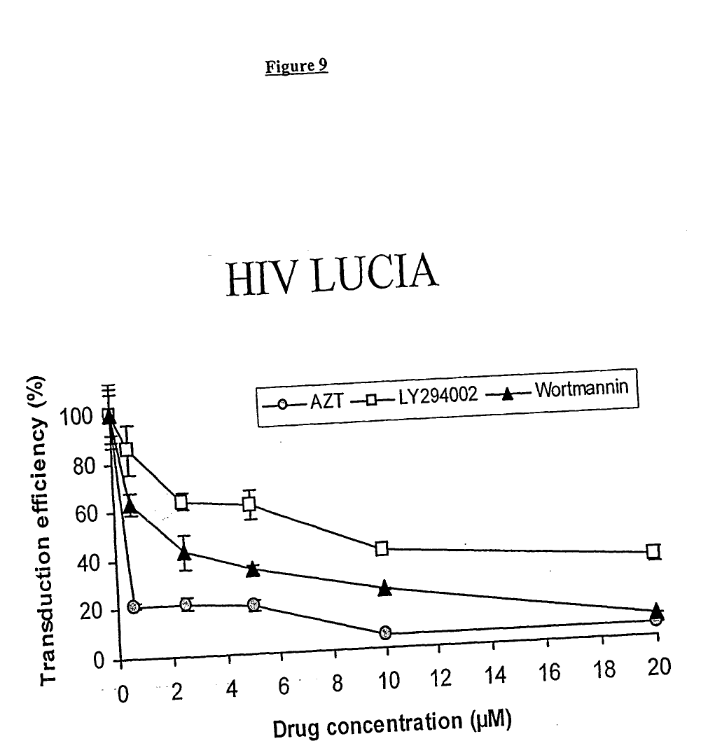

FIG. 9 shows that the DNA-PK inhibitors wortmannin and LY294002 also inhibit the integration of HIV-1 retrovirus in LUCIA assays.

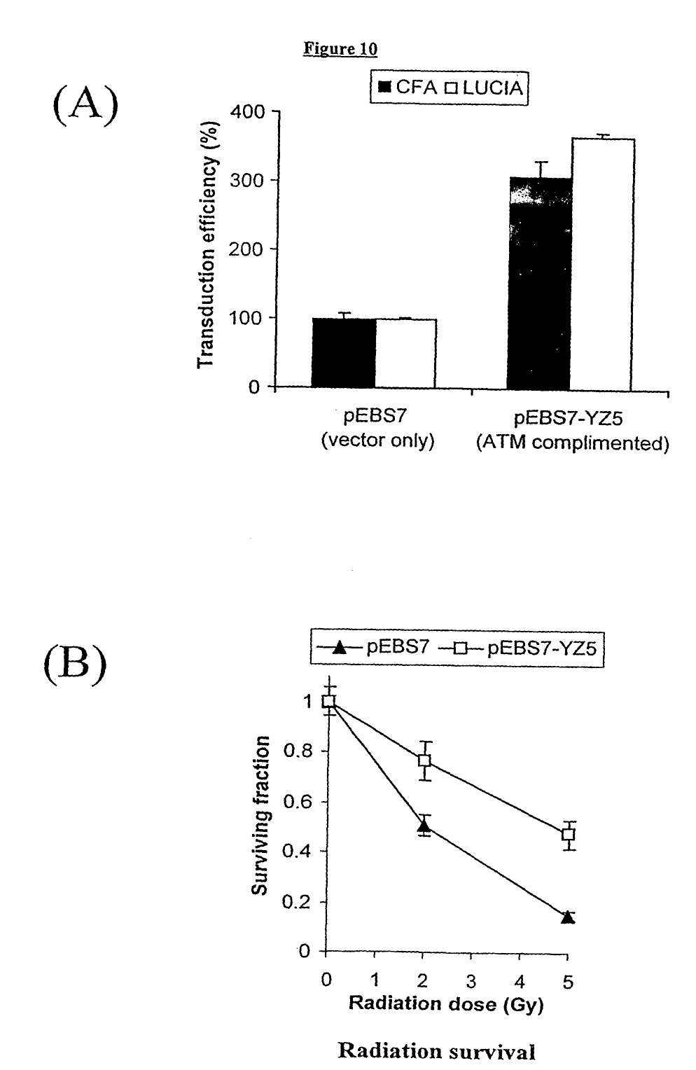

FIG. 10 shows the results of CFA and LUCIA assays demonstrating that efficient retroviral integration can be effectively restored in AT cells through complementation resulting from the stable reintroduction of a functional ATM gene.

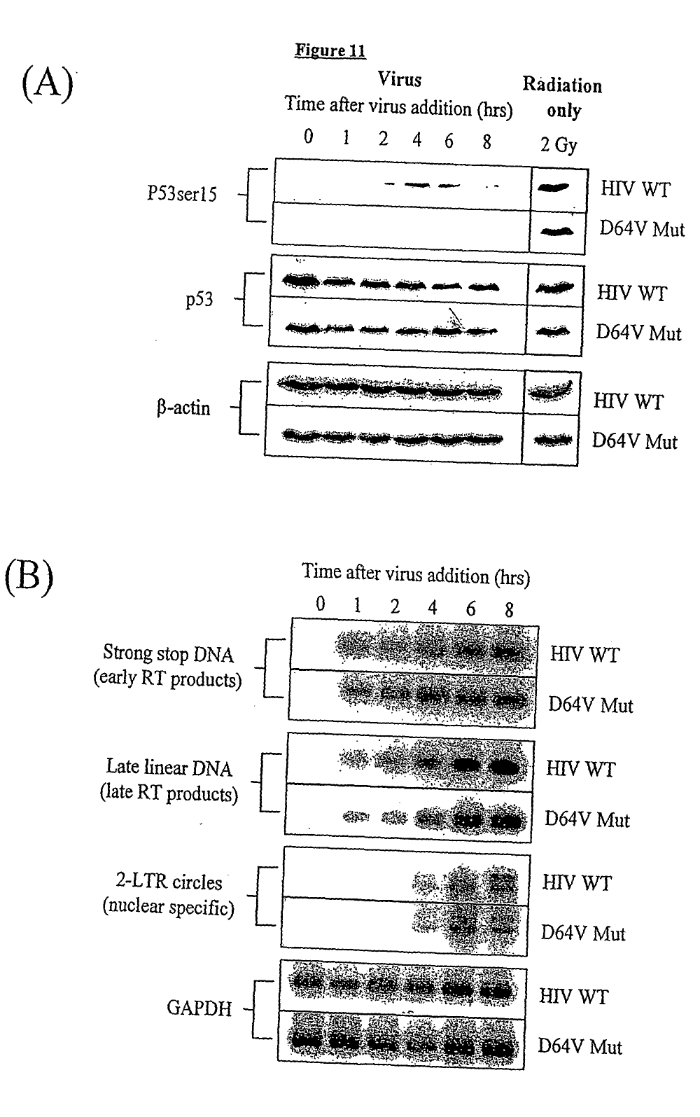

FIG. 11 shows the results of Western (FIG. 11A) and Southern (FIG. 11B) blots which demonstrate ATM-dependent phosphorylation of p53 on serine 15 in response to wild typ HIV-1 infection, but not by an integrase-defective mutant of HIV-1

Murine embryonic stem cell virus (MESV) recombinant retroviral vectors were employed, based upon p50-Mneo (R229) that allows efficient transgene expression in both embyronicf stem (ES) and fibroblast cells (Laker et al., 1998). The retroviral vector capable of expressing luciferase (R229-Luc) was constructed by excising the BglII/XbaI (blunt-ended) fragment containing the SV40 promoter and firefly (Photinus pyralis) luciferase gene from the pGL3-control plasmid (Promega Inc). This was then placed into the BamHI/EcoRI (blunted) sites of the p5O-Mneo (R229) MESV vector. The puromycin expressing retroviral vector (R229-Puro) was constructed by replacing a ClaI/NotI (blunt ended) fragment, containing the neomycin resistance gene (Neo) of p5O-Mneo (R229), with a ClaI/HindIII (blunt ended) fragment that contains the puromycin resistance gene (Puro) of pBABE-puro (Morgenstern et al., 1990).

ES cells were grown in the absence of feeder cells on gelatinized plates in Dulbecco's Modified Eagle Medium (DMEM) with 15% Foetal Bovine Serum (FBS) and supplemented with 500 units/ml Leukaemia Inhibitory Factor (ESGRO, Gibco-BRL), non-essential amino acids and β-mercaptoethanol. Human malignant glioma cells lines MO59K and the DNA-PKcs deficient MO59J cells (Lees-Miller et al., 1995) were grown in DMEM with 10% FBS. Mouse fibroblast cells NIH3T3 and DNA-PKcs mutant SCID cells were grown in DMEM with 10% FBS. Chinese hamster ovary (CHO) cells K1, Ku80 mutant xrs-6 (Jeggo et al., 1983) and XRCC4 mutant XR-1 cells (Stamato et al., 1983) were grown in Minimal Essential medium (MEM) with 10% FBS. Normal human skin fibroblast cells 1BR3 and the DNA ligase IV mutant cell line 180BRM (Riballo et al., 1999) were grown in MEM with 10% FBS and supplemented with non-essential amino acids. Human AT skin fibroblast cells AT5 (BIVA) (Johnson et al., 1999) and NBS skin fibroblasts (Kraakman-van der Zwet et al., 1999) were grown in MEM with 10% FBS and supplemented with non-essential amino acids. ATM complemented AT22IE/pEBS7-YZS and control AT22IE/pEBS7 human fibroblast cells (Ziv et al., 1997) were grown in DMEM with 10% FBS under 100 μg/ml hygromycin selection.

The dualtropic retroviral packaging cell line PT67 was obtained from Clontech Laboratories Inc. and grown in DMEM with 10% FBS.

The NIH3T3/Luc cell line contains a stably integrated and firefly (Photinus pyralis) luciferase expressing R229-Luc provirus. This stable line was constructed by the retroviral transduction of NIH3T3 cells with the R229-Luc retrovirus under identical conditions to the colony forming assays (CFA). Stable luciferase expressing (Luc) cells were generated by selection under 500 mg/ml G418 (Gibco-BRL) and a single cell clone isolated for use.

The retroviral packaging cell line PT67 was used to generate all MESV-based retrovirus-containing supernatants. Stably expressing cell clones were generated by transfection of PT67 cells using Lipofectamine (Gibco-BRL) with either R229Luc or R229-Puro and subsequent selection of G418 resistant (R229-Luc) or puromycin resistant (R229-Puro) cells.

Single, high-titer producing cell clones were isolated for each virus and these were used to generate all retrovirus-containing supernatants. Culture supernatants were filtered through 0.45 μm cellulose acetate membranes (Sartorius) and stored at −80° C. Viral titres were estimated using NIH3T3 cells and CFA (see below). HIV-1 recombinant retroviral stocks were produced in a similar fashion, except that a three-plasmid expression system was used to generate HIV-1 retrovirus-containing supernatants.

For the luciferase integration assays (LUCIA), cells were seeded at 2 to 5×10³ cells per well in 96-well opaque-white tissue culture plates (Corning). For the colony forming unit assay (CFA), cells were seeded at 2 to 5×10⁴ cells per well in 6-well tissue culture plates (Corning). For both assays, cells were allowed to attach for 24 hours. When applicable, the radiosensitizing drugs wortmannin or LY294002 (Alexis Chemicals) were then added to the cells. LUCIA transductions were performed by adding R229-Luc retrovirus containing supernatants at a multiplicity of infection (MOI) of two in the presence of 8 μg/ml polybrene for 6 hours.

Luciferase activity was quantified 48 hours post-transduction on a Packard TopCount-NXT microplate scintillation counter using Steady-Glo luciferase assay reagent (Promega Corp.).

CFA transductions were performed by adding serially diluted R229-Puro retrovirus supernatants at an MOI ranging from 10 to 0.001 in the presence of 8 μg/ml polybrene for 6 hours. 48 hours post-transduction puromycin resistant colonies were selected by changing to fresh medium containing puromycin at 1 to 10 μg/ml. Retroviral integration events were estimated by counting the number of puromycin resistant colonies after 10 to 14 days of puromycin selection.

Standard cytotoxicity/cell viability assays were performed using the CellTiter-96 Aqueous one solution MTS cell proliferation assay (Promega Inc) and using the standard conditions detailed in the manufacturer's instructions.

DNA-PK was purified from HeLa nuclear extract as described previously (Gell et al., 1999). ATM was immunoprecipitated from HeLa nuclear extract using polyclonal antisera raised to the caspase cleavage site region of ATM as described previously (Smith et al., 1999). Kinase assays were performed in 50 mM Hepes, pH 7.5, 50 mM KCl, 4 mM MnCl₂, 6 mM Mg Cl₂, 10% glycerol, 1 mM DTT, 1 mM NaF and 1 mM NaVO4 containing either purified DNA-PK or immunoprecipitated ATM and 1 μg of the substrate GST-p53 (residues 1 to 66). Reactions were pre-incubated at 30° C. for 10 minutes with varying concentrations of wortmannin or LY294002 (for final concentrations see figure). This was followed by the addition of 5 μCi of γ[³³P]-ATP and ATP to a final concentration of 50 μM. Reactions were then incubated for a further 20 minutes at 30° C. before stopping them with SDS-PAGE sample buffer. Proteins were separated by SDS-PAGE and the gels dried before using autoradiography to detect phosphorylated substrate.

For quantification, phosphorylated substrate protein was excised from the dried gels and radioactivity levels counted in the presence of scintillate using a Packard TopCount-NXT microplate scintillation counter.

The standard method for analyzing retroviral integration events is the colony formation assay (CFA) (Stoker, 1993). In this assay, retroviral vectors that carry a selectable drug-resistance gene are used to infect mammalian cells. Genes contained within the LTRs of packaged retroviral vectors are not efficiently expressed unless the virus is successfully integrated into the host genome (Sasaki et al., 1993).

Schematic diagrams of the progenitor retroviral vector R229 based on the murine embryonic stem cell virus (MESV) and the constructed vectors R229-Puro and R229-Luc (see materials and methods) used in the CFA and the LUCIA respectively are shown in FIG. 2(A). The long terminal repeats (LTRs) flank the RNA packaging signal (Ψ), and either the neomycin (Neo^{R}) or puromycin (Puro^{R}) resistance genes. In the case of R229-Luc, the full-length firefly luciferase gene has also been cloned into the vector.

The CFA uses the expression of a drug resistance marker (contained within the retrovirus R229-Puro) and the manual counting of cell colonies after selection to assess the number of retroviral integration events. After incubating the cells with the virus for a few hours, the cells are washed, placed in fresh medium and left to recover for 48 hours. The normal growth medium is then replaced by medium containing the relevant drug. After a further 10 to 14 days of selection, colonies are formed from cells containing integrated provirus and can be counted to provide a quantification of integration events (see FIG. 2B). In order to get an accurate reading, serial dilution experiments must be performed, making this assay both time consuming, laborious and not readily amenable for the analysis of large numbers of samples.

Alternative assays have been described in which reporter genes such as β-galactosidase replace the drug resistance gene (Stoker, 1993). Although faster that CFA, these assays still require time consuming preparation of cells or cell extracts before histochemical or enzymatic analysis. Furthermore these assays are not particularly sensitive or conducive to analysis of large numbers of samples.

A luciferase integration assay employing a retroviral vector bearing the firefly (Photinus pyralis) luciferase gene was designed to provide an assay which was quantitative, faster and less labour intensive than the CFA, but more consistent and sensitive than other reporter based assays. FIG. 2A provides a schematic representation of the R229-Puro retroviral vector, used in our CFAs, and the R229-Luc virus containing the luciferase gene. The outline for the luciferase integration assay (which we have termed LUCIA) is provided in FIG. 2B, where it is compared to the CFA.

Notably, we found that within 48 hours of transduction a significant level of luciferase activity could be detected with as little as 2000 host cells (NIH3T3) at a multiplicity of infection (MOI) of two. FIG. 2B highlights the comparative ease of this assay compared to the standard CFA. Moreover, because so few host cells are required in this assay, we were able to perform the LUCIA in 96-well plates. This allows a large number of repetitions to be carried out concurrently and provides a high throughput analysis of retroviral integration events. LUCIA therefore allows for a much greater high throughput approach than CFA, and provides quantitative results after only 48 hours.

To assess the reproducibility of the LUCIA, we analyzed the effect of mutations in members of the Ku-associated NHEJ pathway using both the conventional CFA and LUCIA. We looked at the ability of mammalian cells containing mutations in the genes for Ku70, Ku80, DNA-PKcs, XRCC4 and ligase IV to support productive retroviral integration. Cells deficient in these various components of the NHEJ pathway were tested for their ability to support retroviral DNA integration (as inferred from transduction efficiency) using both the CFA and the LUCIA. The results of these studies are presented in FIG. 3.

FIGS. 3(A) to 3(D) show results for Ku70 homozygous knockout mouse embryonic stem (ES) cells, Ku80 defective Chinese hamster ovary (CHO) cells xrs6, DNA-PKcs (catalytic subunit) defective mouse severe combined immunodeficient (SCID) fibroblast and human glioma cells M059K, XRCC4 defective CHO cells XR1 and DNA-ligase IV defective human fibroblast cells 180 BRM. Data from both CFA (filled columns) and LUCIA (unfilled columns) are given in these figures as transduction efficiency relative to control wild type (WT) cells.

Consistent with the experiments described by Downs and Jackson (1999) for yeast retrotransposition, mouse embryonic stem (ES) cells containing homozygous deletions for the gene for Ku70 were severely impaired in their ability to support retroviral integration when compared to wild type (WT) ES cells (FIG. 3A). Similarly, Ku80-deficient Chinese Hamster Ovary (CHO) cells were also abrogated in terms of their transduction efficiency compared to the WT K1 cells (FIG. 3B). Daniel et al. (1999) had previously shown that lymphoblastoid and fibroblast cells from the severe combined immunodeficiency (SCID) mouse, which have an inactive mutant form of DNA-PKcs, were impaired in their ability to support retroviral integration. FIG. 3C confirms these results and also demonstrates that DNA-PKcs deficient human glioma MO59J cells are similarly compromised in retroviral transduction when compared to WT M059K cells.

To investigate the role in retroviral integration of XRCC4 and ligase IV directly in mammalian cells, we analyzed the XRCC4 mutant CHO cell line XR1 (Li et al, 1995) and human 180BR cells which have been shown recently to be deficient in DNA ligase IV (Riballo et al, 1999). FIG. 3D clearly demonstrates that impairment of either XRCC4 or ligase IV leads to a marked reduction in the efficiency of retroviral integration. This points to a significant difference in this regard between yeast and mammalian cells. In each of the examples described in FIG. 3, the LUCIA provided results consistent to the standard CFA, but was much easier to perform and yielded results in only a fraction of the time. We looked at the effects of both wortmannin and LY294002, two compounds previously described as inhibiting DNA-PK (Rosenzweig et al., 1997). We utilized both MESV and HIV-1 based retroviruses for this analysis, and also included the retroviral inhibitor 3′-azido-3′-deoxythymidine (AZT) for comparison. AZT is a competitive inhibitor of reverse transcriptase and consequently will prevent retroviral transduction (Mitsuya et al., 1985). NIH3T3 or HELA cells were transduced in MESV based CFA (FIG. 4A) and LUCIA (FIG. 4B) or HIV-1 based LUCIA (FIG. 9) in the presence of various concentrations of the DNA-PK inhibitors wortmannin (filled triangles) and LY294002 (unfilled squares) for 6 hours. Similar treatment with AZT (filled circles) was also carried out for comparison. Data are presented as transduction efficiency relative to untreated cells. For both MESV (FIGS. 4A and 4B) and HIV-1 (FIG. 9), wortmannin and LY294002 inhibit retroviral integration at low micromolar concentrations. What can also be seen is that wortmannin is significantly more efficient than LY294002 in inhibiting retroviral transduction.

Wortmannin has previously been demonstrated to inhibit HIV-1 replication at concentrations similar to that shown here (Sasaki et al., 1995). The study by Sasaki et al. suggested that wortmannin impaired viral budding and release from host cells by inhibiting myosin light chain kinase (MLCK) activity. However, since both the CFA and LUCIA are single cycle virus assays, which only mimic events early in the retroviral life cycle, the observed inhibition by wortmannin cannot be due an effect on MLCK mediated budding and release.

Since this is the first time that wortmannin and LY294002 have been assessed together for their abilities to inhibit productive retroviral integration, we wanted to determine the cytotoxicity of these drugs, at the concentrations used, to ensure this was not the reason for the observed reduction in transduction efficiencies. In addition, we also wanted to ensure that the observations made using the LUCIA were not due to any effects on luciferase expression or on the luciferase assay itself. For this reason, we looked at the effect of adding wortmannin and LY294002 to NIH3T3 cells containing the already integrated R229-Luc provirus.

FIG. 4C presents these control experiments, along with the LUCIA results, for both wortmannin and LY294002. The inhibitory effects of wortmannin and LY294002 in the LUCIA are not due to cellular cytotoxicity (unfilled diamonds ⋄), as determined by MTS formazan dye reduction assays and expressed as the percentage of viable cells remaining after drug treatment because, while there is a slight cytotoxic effect at high concentrations with both compounds, this is not sufficient to account for the drop in retroviral integration events.

Nor is inhibition in the LUCIA the result of repression of luciferase gene expression since neither of these drugs affects the luciferase activity of a stable and integrated R229-Luc provirus (filled squares ▪).

Nor is there any effect of these drugs on the cells containing the R229-Luc provirus that cannot be accounted for by the observed levels of cytotoxicity. Similarly, wortmannin and LY294002 did not inhibit reverse transcriptase activity in vitro, nor did they prevent viral entry, reverse transcription or nuclear accumulation in cells as judged by PCR and southern blot analysis of DNA from retrovirus infected cells.

The effects seen for both wortmannin and LY294002 are therefore due to the targeting of proteins involved in retroviral integration. These results, together with the ability to perform the assay in a 96-well plate format, provide indication that the LUCIA provides a powerful cell-based, high throughput assay to discover small molecule inhibitors of retroviral transduction.

Wortmannin is shown in FIG. 4 to produce a consistently greater level of inhibition of retroviral transduction when compared to LY294002. The ability of wortmannin (20 μM) and LY294002 (20 μM) to inhibit the residual retroviral integration activity of SCID cells that lack functional DNAPKcs was then investigated. If other targets of wortmannin and LY294002 exist in SCID cells, then further inhibition might be observed upon the addition of these compounds. The level of inhibition of retroviral transduction by wortmannin and LY294002 in NIH3T3 and SCID cells compared to untreated control cells was therefore investigated and the results shown in FIG. 5.

LUCIA was performed on both NIH3T3 and SCID cells in the presence of 20 μM wortmannin (filled column) or 20 μM LY294002 (unfilled column). Data are presented as the percentage inhibition of productive retroviral integration events relative to untreated cells. In wild type NIH3T3 cells, it can be seen that while LY294002 inhibits retroviral integration, as in previous experiments, wortmannin demonstrates a greater level of inhibition of retroviral integration than LY294002. In marked contrast, the same experiment in SCID cells clearly demonstrates the inability of LY294002 to bring about inhibition of residual retroviral integration activity. This is not the case for wortmannin, which demonstrates significant inhibition of residual retroviral integration activity in SCID cells.

These data provide indication that wortmannin targets an additional protein(s) to DNA-PK that is involved in the retroviral integration process.

As well as inhibiting DNA-PK kinase activity, wortmannin has also recently been shown to inhibit the function of the related kinase ATM (Moyal et al., 1998; Sakaria et al., 1998; Smith et al., 1999). No one has reported an assessment of the ability of LY294002 to inhibit ATM.

In order to determine if ATM represents the additional target responsible for the differential activity of these two compounds in SCID cells, we looked at the effects of both wortmannin (FIG. 6A) and LY294002 (FIG. 6B) on the kinase activity of ATM. In these biochemical assays, purified DNA-PK and ATM proteins were tested in parallel for their ability to phosphorylate a bacterially expressed GST-p53 substrate in the presence of increasing concentrations of wortmannin or LY294002 (see Materials and Methods). Kinase reactions containing either purified DNA-PK or immunoprecipitated ATM and GST-p53 substrate (p53 residues 1 to 66) were performed in the presence of varying concentrations of wortmannin or LY294002 as indicated in FIG. 6.

FIG. 6A shows that phosphorylation of the p53 substrate by DNA-PK is almost completely inhibited in vitro at concentrations of wortmannin of 0.25 μM. ATM kinase activity is also abolished by wortmannin, as previously described (Moyal et al., 1998; Sakaria et al., 1998; Smith et al., 1999), although this occurs at the higher concentration of 5 μM. Notably, the compound LY294002, while completely inhibiting DNA-PK activity at a concentration of 50 μM as previously described (Izzard et al., 1999), shows virtually no inhibitory capacity against ATM, even at concentrations far greater than those inhibiting DNA-PK activity. These data demonstrate the differential effects of wortmannin and LY294002 on ATM. They also provide a potential explanation for the observed differences between wortmannin and LY294002 in inhibiting residual retroviral integration in SCID cells (see below).

The results presented in FIGS. 4, 5 and 6 provide indication that differences in the abilities of wortmannin and LY294002 to inhibit retroviral integration can be attributed to the targeting of the ATM protein. Targeting ATM and/or other components of the ATM-dependent DNA damage signalling pathway is therefore shown to produce distinct effects which are independent of those caused by the inhibition of DNA-PK.

We performed the LUCIA with both mouse ES cells deficient in ATM (Xu et al., 1996), with human fibroblasts derived from an ataxia telengiectasia (AT) patient (Gilad et al., 1996) and with AT cells in which its ATM defect has been complemented by stable reintroduction of a functional ATM gene (Ziv et al., 1997).

ATM homozygous knockout mouse ES cells and a human fibroblast cell line derived from an ataxia-telangiectasia (AT) patient (AT5 BIVA) were tested in the LUCIA and found to be impaired for their ability to productively integrate retroviral DNA. Data are presented in FIG. 7 as transduction efficiency relative to control wild type (WT) cells. An analysis of the ability of 20 μM wortmannin (filled columns) or 20 μM LY294002 (unfilled columns) to inhibit retroviral integration events in WT cells and those containing no functional ATM protein is shown in FIG. 7B. Data are presented as the percentage inhibition of productive retroviral integration events relative to untreated cells. At 20 μM, both wortmannin and LY294002 show comparable inhibitory effects on retroviral integration in ATM knockout (−/−) and mutant (AT5) cells. This is in contrast to the differential effects previously seen in SCID cells.

FIG. 7A demonstrates that retroviral transduction is significantly impaired in cells deficient in the ATM protein when compared to WT ATM control cells, which provides indication that the ATM protein is required for efficient retroviral integration. The results provided thus far indicate that the ability of wortmannin, but not LY294002, to inhibit residual retroviral integration in SCID cells (FIG. 5) may be attributed to the inhibition of ATM activity and not to other properties of the compounds themselves, such as their ability to penetrate the cell membrane or persist in an active form inside the cell. Further evidence to support this is presented in FIG. 7B which reveals essentially no difference between the ability of the two compounds to inhibit retroviral integration with either ATM deficient mouse ES cells or the human AT5 fibroblasts.

Retroviral transductions were also performed on ATM deficient AT22IE cells in which the defect has been complemented by stable reintroduction of a functional ATM gene (AT22IE/pEBS7-YZ5; Ziv et al., 1997). Efficient retroviral transduction was effectively restored in the complemented AT22IE/pEBS7-YZ5 cells when compared to the vector only AT22IE/pEBS7 cells (see FIG. 10A). For comparison, the X-ray radiation cell survival curves for AT22IE/pEBS7-YZ5 and AT22IE/pEBS7 cells are also shown (FIG. 10B). It can be seen that there is good correlation between the relative level of complementation for retroviral transduction and X-ray-induced DNA damage sensitivity. There were no significant differences in viral entry, reverse transcription or nuclear entry between AT22IE/pEBS7 and AT22IE/pEBS7-YZ5 cells, as judged by PCR analysis of viral DNA intermediates, providing indication that it is the specific impairment of the later steps of the retroviral integration process that is responsible for the observed differences between these two cell lines.

Further evidence that it is the retroviral integration step that mediates an ATM-dependent DNA damage response was obtained by comparing the ability of both wild type and D64V integrase-deficient mutants of HIV-1 to stimulate ATM kinase activity. Activated ATM can be indirectly measured through the use of specific antibodies that recognize ATM-dependent phosphorylation of p53 residue serine 15 (Ser15). FIG. 11A shows that infection of mammalian cells with wild type HIV-1 retrovirus induces an ATM-dependent phosphorylation of p53 Ser15, as does the previously observed response to ionizing radiation induced DNA damage (Siliciano et al., 1997). However, this specific ATM-dependent phosphorylation event was abolished when using the D64V integrase-defective mutant virus, consistent with the idea that retroviral infection stimulates a specific ATM and DNA damage-dependent phosphorylation that requires a functional integrase protein. Reprobing of the blot for total p53 protein levels and β-actin indicated no significant difference in the loading of cellular proteins (FIG. 11A). PCR analysis of DNA from cells at various times during retroviral infection also showed no significant differences in virus entry and reverse transcription (early and late RT products) or nucleic entry (2-LTR circle products) between wild type and integrase D64V mutant viruses (FIG. 11B). It can be seen from the early RT products that the integrase D64V mutant virus reverse transcribes as efficiently as wild type virus. Since the integrase D64V mutant virus fails to promote p53 Ser15 phosphorylation, these data provide indication that the presence of un-integrated viral dsDNA, in itself, does not induce an ATM response.

Together these results provide evidence that the activation of ATM by retroviral infection is an integration-dependent process that occurs as a result of the ensuing DNA damage and that efficient completion of integration process is dependent upon this ATM activity.

In response to chromosomal DNA damage an ATM-dependent DNA damage signalling pathway induces a transient cell cycle arrest to allow efficient DNA repair and cell survival (see Lavin and Shiloh, 1997 for review). Downstream targets and effectors of ATM protein function have been shown to include Chk2 (Matuoka et al., 1998; Blastine et al. 1999; Chaturvedi et al. 1999) BRCA1, and recently NBS1 (Lim et al., 2000).

LUCIA was performed on human colon cells with a defective Chk2 protein (Bell et al., 1999), and on NBS1 protein defective human fibroblast cells derived from a Nijmegen breakage syndrome (NBS) patient (Kraakman-van der Zwet et al., 1999). Human colon cancer cells (HCT15) containing a missense mutation in the Chk2 gene (Bell et al., 1999) were shown to be impaired in their ability to support productive retroviral integration, when compared to the human colon cancer cells (HCT116) that are WT for Chk2 (FIG. 8(A)). NBS1 (p95), a component of the human Mrell-Rad50-containing DNA repair complex and the cause of Nijmegen breakage syndrome (NBS), was also shown to play a role in retroviral integration (FIG. 8(B)). These data are presented as transduction efficiency relative to control wild type (WT) cells in FIG. 8 and show that Chk2 and the NBS1 protein, both downstream effectors of ATM cell cycle checkpoint function, are required for efficient retroviral transduction.

The cell cycle regulatory protein p53 (Ko and Prives, 1996) is a known downstream target of ATM activity (Kastan et al., 1992). To investigate the possible role of p53 in retroviral integration, we carried out the LUCIA in two osteosarcoma cell lines that differ in their p53 status. U2OS cells contain functional endogenous p53 protein, whilst SOAS2 cells completely lack the p53 protein.

FIG. 8C demonstrates that while U2OS cells can support productive retroviral integration events, SOAS2 cells do not. To investigate these initial results further, we carried out a transient transfection of U2OS cells, 24 hours prior to transduction with the R229-Luc virus, with either a plasmid containing the MDM2 cDNA driven by a CMV promoter or, as a control, the empty CMV vector lacking MDM2 sequences. Retroviral integration events were then determined by LUCIA 24 hours after the addition of virus and the results are presented in FIG. 5D.

The MDM2 gene product down-regulates p53 activity and promotes the degradation of cellular p53 protein (Lane and Hall, 1997). U2OS cells transiently transfected with the CMV-MDM2 plasmid demonstrated a reduction in cellular p53 as shown by western-blot analysis using the p53-specific monoclonal antibody DO-1 (Santa Cruz). This reduction in endogenous p53 protein levels was accompanied by a reduction in retroviral integration events as compared with cells transfected with the control CMV vector.

The dependence of retroviral integration on proteins involved in the ATM mediated DNA damage signaling pathway is demonstrated by the impaired ability of cells defective in Chk2, NBS1 and p53 proteins to support retroviral integration when compared to WT control cells. These findings provide indication that proteins downstream of Chk2, NBS1 and p53 play a role in retroviral integration.

### REFERENCES

1 Barnes et al. (1992). Cell 69. 495-503.

2 Bell et al. (1999). Science 286. 2528-2531.

3 Bentley et al. (1997). Biol. Chem. 378 (11) 1267-1274.

4 Blasina et al. (1999). Curr Biol 9. 1-10.

5 Boer et al. (1990). Biochem Genetics 28. 299-308.

6 Brown PO. (1990). Curr Top Microbiol Immunol 157. 19-48.

7 Bushman et al. (1990). Science 249. 1555-1558.

8 Chaturvedi et al. (1999). Oncogene 18. 4047-4054

9 Chow SA. (1997). Methods 12. 306-312.

10 Craigie et al. (1990). Cell 62. 829-837.

11 Critchlow SE and Jackson SP. (1998). Trends in Biochemical Sciences 23. 394-398.

12 Daniel et al. (1999). Science 284. 644-647.

13 Downs et al. (1999). Mol Cell Biol 19. 6260-6268.

14 Essers et al. (1997). Cell 89. 195-204.

15 Friedberg et al. (1995). ASM Press. 698

16 Gaken et al. (1996). J Virol 70. 3992-4000.

17 Gell et al. (1999). Nucleic Acid Res 27. 3494-3502.

17a Gilad et al. (1996). Hum Mol Genet. 5.433-439.

18 Gu, Y. et al. (1997). Proc Natl Acad Sci USA 94. 8076-8081.

19 Hazuda et al. (2000). Science 287. 646-650. Hirt B. (1967). J Mol Biol 26. 365-369.

21 Izzard et al. (1999). Cancer Res 59. 2581-2586.

22 Jeggo et al. (1983). Mutation Res 112. 313-327.

23 Kastan et al. (1992). Cell 71. 587-597.

24 Katz et al. (1990). Cell 63. 87-95. Kraakman-van der Zwet et al. (1999). Mutat. Res 434. 17-27

26 Laker et al. (1998). J. Virol. 72.339-348.

26a Lane and Hall. (1997). Trends Biochem. Sci. 22. 372-374.

27 Lavin MF and Shiloh Y (1997). Annu Rev Immunol 15. 177-202.

28 Lees-Miller et al. (1995). Science 267. 1183-1185.

29 Li et al. (1995). Cell 83. 1079-1089.

30 Liang et al. (1996). Proc Natl Acad Sci USA 93(17) 8929-8933

31 Lim et al. (2000). Nature 404. 613-617.

32 Lindahl et al. (1995). Trends in Biochemical Sciences 20. 405-411.

33 Matsuoka et al. (1998). Science 282. 1893-1897.

34 Melissa et al. (1998). J Biol Chem 273. 30069-30072.

35 Mitsuya et al. (1985). Proc Natl Acad Sci USA 82. 7096-7100.

36 Morgenstern et al. (1990). Nucleic Acids Res 18. 3587-3596.

37 Petukhova et al. (1998). Nature 393. 91-94.

38 Pommier et al. (1999). Adv Virus Res 52. 427-458.

38a Ko and Prives. (1996). Genes. Dev. 10. 1054-1072.

39 Riballo et al. (1999). Current Biology 9. 698-702.

40 Rosenweig et al. (1997). Clin Cancer Res 3. 1149-1156.

41 Sambrook et al. (1989). Molecular Cloning: A laboratory manual. 2^{nd} edition. Cold Spring Harbour Press.

42 Sasaki et al. (1993). J Virol 67. 1169-1174.

43 Sasaki et al. (1995). Proc Natl Acad Sci USA 92. 2026-2030.

44 Satoh et al. (1994). Biochemistry 33. 7099-7106

44a Siliciano, J. D. et al. (1997) Genes Dev. 11. 34713481.

45 Smith et al. (1999). Mol Cell Biol 19. 6076-6084

46 Sobol et al. (1996). Nature 379. 183-186.

47 Sonoda et al. (1999). Molecular and Cellular Biology 19. 5166-5169.

48 Stamato et al. (1983). Somatic Cell Genet 9. 165-173

49 Stoker A W. (1993). Molecular Virology: A Practical approach. Oxford University Press. 171-197.

50 Tan et al. (1999). Current Biology 9. 325-329

51 Tanaka et al. (2000) Nature 404 42-49

52 Taylor et al. (1975). Nature 258 (5534) 427-429

53. Xu et al. (1996). Genes Dev 10. 2401-2410

54. Ziv et al. (1997) Oncogene 15. 159-67

1. A method of screening for an agent which is an inhibitor of retrovirus and/or retrotransposon activity, which method comprises:
providing an ATM-dependent DNA damage signalling pathway;
exposing the pathway to a test substance under conditions which would normally lead to the activation of the ATM-dependent DNA damage signalling pathway;
determining an end-point indicative of activation of the ATM-dependent DNA damage signalling pathway, whereby inhibition of that end-point indicates inhibition of the ATM-dependent DNA damage signalling pathway by the test substance and whereby test substance which inhibits the ATM-dependent DNA damage signalling pathway is identified; and
determining ability of said test substance which inhibits the ATM-dependent DNA damage signalling pathway to inhibit retrovirus and/or retrotransposon activity.

2. A method of screening for an agent which is an inhibitor of retrovirus and/or retrotransposon activity, which method comprises:
providing a component of a ATM-dependent DNA damage signalling pathway or a fragment of such component;
exposing said component or fragment to a test substance;
determining interaction between said component or fragment and the test substance, whereby test substance which interacts with said component or fragment is identified; and
determining ability of said test substance which interacts with said component or fragment to inhibit retrovirus and/or retrotransposon activity.

3. A method of screening for an agent which is an inhibitor of retrovirus and/or retrotransposon activity, the method comprising:
providing first and second substances, the first substance comprising a first component of a ATM-dependent DNA damage signalling pathway or a peptide fragment of said first component wherein said first component or fragment of said first component is able to bind a second component of the ATM-dependent DNA damage signalling pathway, the second substance comprising said second component of the ATM-dependent DNA damage signalling pathway or a peptide fragment of said second component wherein said second component or fragment of said second component is able to bind said first component, under conditions in which the first and second substances normally interact;
exposing the first and second substances to a test compound;
determining interaction between the first and second substances in the presence of the test compound, whereby test compound which disrupts interaction between the first and second substances is identified; and
determining the ability of said test compound which disrupts interaction between the first and second substances to inhibit retrovirus and/or retrotransposon activity.

4. A method according to any one of claims 1 to 3 further comprising formulating said agent into a composition comprising at least one additional component.

5. A method according to claim 4 which comprises combining said agent with a pharmaceutically acceptable excipient.

6. A method according to any one of claims 1 to 5 further comprising providing said agent to a cell to inhibit retrovirus and/or retrotransposon activity.

7. A method according to claim 6 wherein said cell is not part of a human or animal body.

8. A method according to any one of claims 1 to 5 further comprising use of said agent in the manufacture of a medicament for inhibiting retrovirus and/or retrotransposon activity.

9. An inhibitor of retrovirus and/or retrotransposon activity obtained using a method according to any one of claims 1 to 3.

10. Use of an inhibitor of the ataxia telangiectasia mutated (ATM)-dependent DNA damage signalling pathway in the manufacture of a medicament for treatment to inhibit retrovirus and/or retrotransposon activity, with the proviso that the inhibitor is not wortmannin.
